(19) United States
(12) Patent Application Publication
(10) Pub. No.: US 2009/0149638 A1
(43) Pub. Date: Jun. 11, 2009

(54) SYSTEMS AND METHODS FOR PURIFYING PROTEINS

(76) Inventors: Arthur C. Ley, Newton, MA (US); Jie Chen, Sudbury, MA (US)

Correspondence Address: LOWRIE, LANDO & ANASTASI, LLP, ONE MAIN STREET, SUITE 1100, CAMBRIDGE, MA, 02142, US

(21) Appl. No.: 12/238,797

(22) Filed: Sep. 26, 2008

Related U.S. Application Data
(60) Provisional application No. 60/977,155, filed on Oct. 3, 2007.

(51) Int. Cl. C07K 1/34; C12M 1/00

(52) U.S. Cl. 530/412; 435/283.1

(57) ABSTRACT

Described herein are novel systems and downstream protein purification (DSP) processes that provide high quality product rapidly, and on a large scale. Many of the processes enable one chromatography step to follow another chromatography step without an intermediate ultrafiltration/diafiltration (UFDF) step. These optimized processes allow for automation on the manufacture plant floor, permitting the use of a multi-cycling strategies that can utilize smaller, less expensive columns. The processes can provide considerable advantage on production efficiency, cost saving and on waste disposal.

### CROSS-REFERENCE TO RELATED APPLICATIONS

This application claims priority to U.S. Application Ser. No. 60/977,155, filed on Oct. 3, 2007. The disclosure of the prior application is considered part of (and is incorporated by reference in) the disclosure of this application.

### TECHNICAL FIELD

This invention relates to systems and methods of purifying proteins, such as antibodies.

### BACKGROUND

The large-scale production of pharmaceutical-grade monoclonal antibodies (mAbs) is a complex manufacturing process, often with multiple chromatography and filtration steps designed to satisfy stringent regulatory requirements. With the increasing success of therapeutic mAbs [1], focus has generally turned to improving process efficiencies, product quality, and to decreasing costs [2-4,6].

The past decade has brought improvements both in the yields of the upstream processes for mAb production and in the analytical technologies to characterize impurities and contaminants [2-6]. An industry-wide drive for high throughput at a low cost is reshaping mAb purification process development strategies [2-4,6,7].

Hydrophobic interaction chromatography (HIC) is a major “polishing step” in the purification process of IgG-based products, and is known for its capability to remove aggregated forms of antibody [8-14]. Although HIC is a powerful tool in mAb purification processes, process scientists understand its central limitations. Sufficient binding of mAb proteins to HIC resins is usually achieved with increasing salt concentrations in the binding buffers and the elution product from the HIC purification step may contain appreciable amounts of salt, which can complicate sample manipulations and process flow transitions during large-scale manufacture since most other chromatographic techniques used for mAb purification including Ion Exchange and Hydroxyapatite require binding mAb at low ionic strength conditions [4,10,11].

Other chromatographic techniques for purifying proteins are described in references [15-21].

### SUMMARY

Generally, this invention relates to systems and methods of purifying proteins, such as antibodies, e.g., monoclonal antibodies and fragments thereof.

In one aspect, the invention features protein purification systems that include one or more columns, each including an adsorbent therein. The protein purification systems are capable of accepting a culture having a protein concentration of greater than about 5 g/L, and are also capable of purifying the protein to an extent of greater than about ninety-five percent, as measured using SEC-HPLC, with an overall yield of greater than about forty percent.

In another aspect, the invention features protein purification systems that include one or more columns, each including an adsorbent therein. The protein purification systems are capable of processing greater than about 200 L per hour of a culture having a protein concentration of greater than about 5 g/L.

In another aspect, the invention features protein purification systems that include one or more columns, each including an adsorbent therein. Each column includes less than about 250 L of adsorbent, and the protein purification systems are capable of accepting a culture having a protein concentration of greater than about 5 g/L.

In another aspect, the invention features methods of purifying proteins that include providing a culture that includes a protein; flowing the culture, e.g., clarified culture, through a first column that includes a first adsorbent to provide a first eluate that includes the protein; and flowing the first eluate, or a concentrated or a diluted form thereof, through a second column that includes a second adsorbent without prior filtration, e.g., difiltration or ultra filtration, of the first eluate, or the concentrated or the diluted form thereof, to provide a second eluate including the protein. For example, the method may further include flowing the second eluate, or a concentrated or a diluted form thereof, through a third column that includes a third adsorbent without prior filtration, e.g., difiltration or ultra filtration, of the second eluate, or the concentrated or the diluted form thereof, to provide a third eluate including the protein. For example, the culture can be provided by a recombinant cell, e.g., a CHO cell.

Aspects and/or embodiments may have one or more of the following advantages. The unique design for MEP elution allows for better separation resolution to provide purer product. The optimal process flow design platform allows for the elimination of an intermediate UFDF process and also provides benefits for manufacture plant automation plan. The processes and systems described herein are scalable and capable of being operated on a high-throughput and continuous basis. The processes are capable of handling high titer concentrations, e.g., concentrations of about 5 g/L, greater than about 5 g/L, e.g., greater than about 6, about 7, about 8, about 9, about 10, about 15, about 25 or even greater than about 50 g/L. For example, some of the systems can process greater than about 200 L culture per hour, e.g., greater than about 400 L, about 600 L, about 800 L or even greater than about 1500 L per hour. The processes can offer an equivalent purity protein or even a higher purity protein product, e.g., as compared to known purification techniques, at a reduced cost. The amount of adsorbents, such as resins, overall can be greatly reduced, e.g., by 25 percent, 50 percent, 75 percent or even 90 percent. In some systems, the multiple-column processes do not require filtering, e.g., via ultrafiltration/diafiltration, and/or other significant sample manipulations between each pair of columns. Not filtering and/or diluting between column pairs can enable higher throughput and can allow for a continuous process and/or multiple passes through the systems to increase purity and/or efficiency. Not filtering and/or diluting can also enable smaller columns and/or reduce process time, which can lower the usage of expensive adsorbents and/or can lower the overall cost of the processes. The higher throughput systems described herein can make desirable and life-saving therapeutics and diagnostics available to patients at a reachable cost.

In some aspects, the ProA→MEP→CHT/AEX DSP design allows for one or more of the following advantages: the elimination of intermediate UFDF processes, which allows for increased production efficiency and/or cost savings; better separation resolution and purer monomer antibody products when eluting antibody products with a dominant HIC strategy in the mix mode (e.g., dual mode) MEP resin; chromatography purification steps can be easily streamlined and/or automated at manufacturing plant floors when using the mix mode MEP step as a post ProA purification unit; and/or the use of smaller columns and/or multi-cycling strategies for downstream production using streamlined and automated production processes can provide solutions for downstream processes at manufacturing plants to adapt to increasing (e.g., high) production rates from upstream mammalian cell fermentation process optimizations.

In some aspects, use of the methods described herein provide (e.g., result in) a purer antibody product, e.g., as compared to an antibody purified by known (e.g., conventional) methods of purification (e.g., downstream purification platforms that use only ProA and/or cation/anion exchange chromatography). For example, a given purified antibody product can have lower levels of aggregates (e.g., high molecular weight aggregates; HMW), lower levels of leached ProA (e.g., ProA ppm) and/or lower levels of host cell contaminating proteins (e.g., HCP ppm) (e.g., CHO cell protein contaminates (e.g., CHO HCP ppm)) as compared to an antibody purified by known (e.g., conventional) methods of purification, e.g., such as methods that utilize a UFDF step and/or methods that include diluting eluates prior to applying the eluate to a subsequent column (e.g., to dilute a salt concentration of the eluate), or downstream purification platforms that use only ProA and/or cation/anion exchange chromatography.

The following abbreviations used herein have the following meanings: LC, liquid chromatography; HPLC, high pressure liquid chromatography; mAb, monoclonal antibody; ProA, Protein A; CEX, cation exchange chromatography; AEX, anion exchange chromatography; HIC, hydrophobic interaction chromatography; HCIC, hydrophobic charge induction chromatography; MEP, mercapto-ethyl-pyridine; CHT, ceraminc hydroxyapatite; SEC, size exclusion chromatography; UFDF, ultrafiltration/diafiltration; USP, upstream processing; DSP, downstream processing (purification); CHO, Chinese hamster ovary cells; LMW, low-molecular weight; and HMW, high-molecular weight; ppm, parts per million.

Examples of upstream processes include those that produce a product, e.g., a bulk product, e.g., in unpurified form. For example, host cell expression systems used to recombinantly express a protein (e.g., antibody) product of interest are considered to be upstream processes. Downstream processes (e.g., purification processes) are then performed to extract and/or purify the product of interest that results from the upstream process. Additional examples of upstream process are shown in FIG. 1 below line 9; and additional examples of downstream processing are shown in FIG. 1 above line 9.

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)2, a Fd fragment, a Fv fragments, and dAb fragments) as well as complete antibodies.

Exemplary antibodies that can be subjected to the described process system include the antibodies described in U.S. Publication No.: 20060057138 such as DX-2240, U.S. Publication No.: 20070004910 such as DX-2300 and U.S. Publication No.: 20070217997 such as DX-2400, the contents of which are incorporated herein by reference.

The described process system can be used to purify a protein (e.g., an antibody), e.g., a recombinant protein (e.g., a recombinant antibody), from cell culture. The cells can be eukaryotic or prokaryotic. Examples of eukaryotic cells include yeast, insect, fungi, plant and animal cells, especially mammalian cells. Suitable mammalian cells include any normal mortal or normal or abnormal immortal animal or human cell, including: monkey kidney CV1 line transformed by SV40 (COS-7, ATCC CRL 1651); human embryonic kidney line (293) (Graham et al., J. Gen. Virol. 36:59 (1977)); baby hamster kidney cells (BHK, ATCC CCL 10); Chinese Hamster Ovary (CHO) cells, e.g., DG44, DUKX-V11, GS-CHO (ATCC CCL 61, CRL 9096, CRL 1793 and CRL 9618); mouse sertoli cells (TM4, Mather, Biol. Reprod. 23:243 251 (1980)); monkey kidney cells (CV1 ATCC CCL 70); African green monkey kidney cells (VERO-76, ATCC CRL 1587); human cervical carcinoma cells (HeLa, ATCC CCL 2); buffalo rat liver cells (BRL 3A, ATCC CRL 1442); human lung cells (W138, ATCC CCL 75); human liver cells (Hep G2, HB 8065); mouse melanoma cells (NSO); mouse mammary tumor (MMT 060562, ATCC CCL51), TRI cells (Mather, et al., Annals N.Y. Acad. Sci. 383:44 46 (1982)); canine kidney cells (MDCK) (ATCC CCL 34 and CRL 6253), HEK 293 (ATCC CRL 1573), WI-38 cells (ATCC CCL 75) (ATCC: American Type Culture Collection, Rockville, Md.), MCF-7 cells, MDA-MB-438 cells, U87 cells, A127 cells, HL60 cells, A549 cells, SP10 cells, DOX cells, SHSY5Y cells, Jurkat cells, BCP-1 cells, GH3 cells, 9L cells, MC3T3 cells, C3H-10T1/2 cells, NIH-3T3 cells and C6/36 cells.

All publications, patent applications, patents, and other references mentioned herein are incorporated by reference herein in their entirety for all that they contain.

Other features and advantages will be apparent from the following detailed description, and from the claims.

### DESCRIPTION OF DRAWINGS

FIG. 1 is a schematic diagram of a generalized process for making and purifying antibodies on a large scale.

FIG. 2 is an LC chromatogram of a DYAX mAb DX2300-rich eluate obtained by flowing a culture containing the mAb through a ProA column.

FIG. 3 is an LC chromatogram of a DYAX mAb DX2300-rich eluate obtained by passing the eluate of FIG. 2 through an MEP column.

FIG. 4 is an LC chromatogram of a DYAX mAb DX2300-rich eluate obtained by passing the eluate of FIG. 3 through a CHT column.

FIG. 5 is a SEC-HPLC chromatogram of the DYAX mAb DX2300-rich eluate of FIG. 2.

FIG. 6 is a SEC-HPLC chromatogram of the DYAX mAb DX2300-rich eluate of FIG. 3.

FIG. 7 is a SEC-HPLC chromatogram of the DYAX mAb DX2300-rich eluate of FIG. 4.

FIGS. 8A and 8B. FIG. 8A is a LC chromatogram of a DYAX mAb DX2400-rich eluate obtained by passing the ProA eluate through MEP column using dual separation strategies. FIG. 8B is a table of product purity analysis.

### DETAILED DESCRIPTION

Described herein are novel downstream protein purification (DSP) processes that provide high quality product rapidly, and on a large scale (e.g., capable of processing greater than about 200 L of culture per hour). Many of the processes enable one chromatography step to follow another chromatography step without an intermediate ultrafiltration/diafiltration (UFDF) step. These optimized processes allow for automation on the manufacture plant floor, permitting the use of multi-cycling strategies that can require smaller, less expensive columns. The processes can provide considerable advantages on production efficiency, cost savings and/or on waste disposal.

Studies on unit operation for mix mode resins, such as Hydrophobic Charge Induction Chromatography resin MEP, ceramic hydroxyapatite resin CHT and CAPTO™ Adhere, unit operation in monoclonal antibody purification application and separation mechanism were performed and systematic downstream purification (DSP) platform studies were designed and conducted for mAbs DX2240, DX2300 and DX2400. The DSP platform designs with mix mode resin, MEP, as post ProA intermediate purification step, have significant process flow benefits, which enable the chromatography step elution product pool to feed subsequent chromatography steps one after another with no requirement for an intermediate ultrafiltration/diafiltration (UFDF) process or large volume dilution (e.g., greater than a 1:1 dilution; e.g., the process platform design described herein allows less than 1:1 dilution). By using a mix mode MEP chromatography step as a second intermediate purification process, it not only can facilitate process flow transition but it also is able to provide significant separation benefit through manipulation of its HIC/IEX dual mode elution pattern.

The invention also includes the unique elution strategy of using solely HIC mode to elute IgG monomer and retain aggregates and other impurities until later ion exchange mode discharge by the resin manufacture's common recommendations. The unique platform designs ProA→MEP→CHT and ProA-MEP-AEX/CAPTO™ Adhere can provide not only comparable or better product quality (e.g., than known purification methods) but also less efforts for process development and friendly engineer design potential for manufacture automation. In particular, ProA-MEP-CHT is a platform that can often deliver better removal of aggregates compared to conventional mAb downstream purification platforms that use only ProA and/or cation/anion exchange chromatography. Therefore, the ProA-MEP-CHT platform provides advantages when loading material having higher aggregate levels, e.g., materials that contain antibodies. Because of the optimized process flow, the DSP designs described herein allow for simple automation design on the manufacture plant floor, which permits the use of smaller columns and/or multi-cycling strategies in continuous processes for mAb product downstream production. These DSP designs provide a strategy for resolving upstream high productivity challenges and results in considerable advantages on downstream purification production efficiency and cost saving.

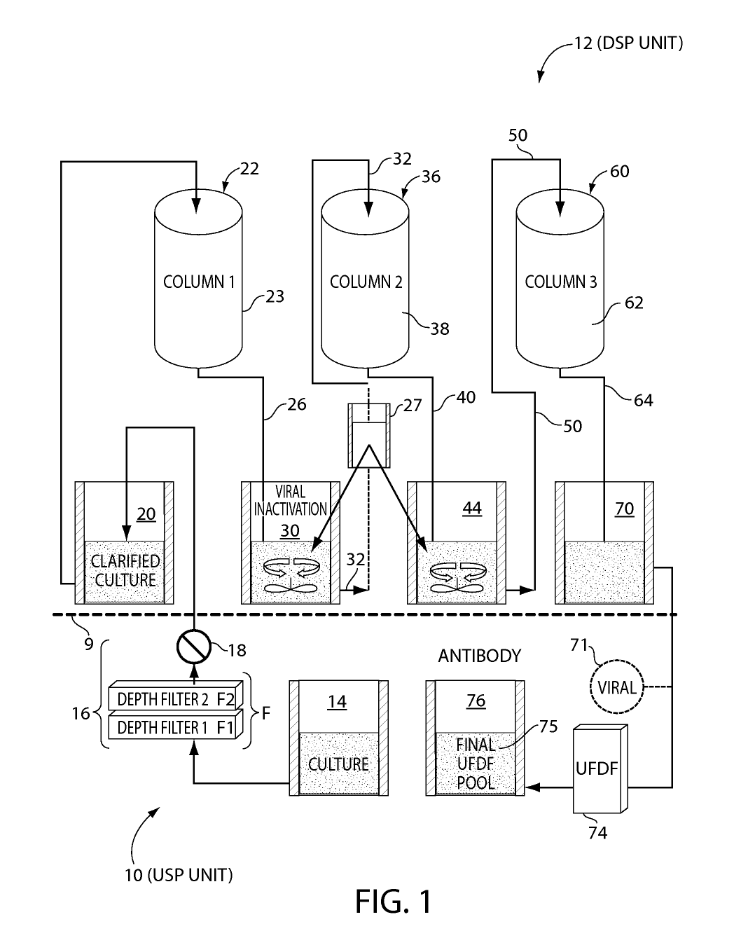

Referring to FIG. 1, a system for the large scale production of antibodies includes an upstream processing unit 10 (USP, below line 9) for making crude antibody and a downstream processing unit 12 (DSP, above line 9) for purifying the crude antibody. The USP unit 10 includes a culture forming unit 14 and a culture clarifying unit 16, which can include a plurality of depth filters F (shown with two filters, F1 and F2 in FIG. 1), and, optionally, one or more ultrafiltration/diafiltration units 18 (shown with one if FIG. 1). For example, the depth filters can be in the form of membranes having pores from <0.1 to about 8 microns, e.g., about 2 to about 5 microns. In some embodiments, the pores are greater than 1 micron. In some embodiments, the pores are greater than about 1 micron. In some embodiments, the pores are less than 1 micron. In some embodiments, the pores are about 0.2 microns.

The USP unit 10 provides a clarified culture that includes an antibody of interest to a holding tank 20. The clarified culture, or a concentrated or diluted form of the culture, is transferred to a first column 22 that includes a first adsorbent 23. The clarified culture flows through the first column to provide a first eluate 26 that includes the antibody of interest. Optionally, elution of the first elute 26 can be performed under acidic conditions and the first elute can be maintained in holding tank 30 under the acidic conditions, e.g., for 1-2 hours, to inactivate viral load. The first elute can then be neutralized, e.g., using Tris buffer from tank 27 to provide a neutralized material 32.

Neutralized material 32 that includes the antibody of interest can then be transferred to a second column 36 that includes a second adsorbent 38, optionally, without prior filtration and/or other manipulation (e.g., dilution) of the neutralized material. The unfiltered and neutralized material flows through the second column to provide a second eluate 40 that includes the protein of interest. As shown, elution of the second eluate 40 optionally can be performed into a holding tank 44. Here, the second eluate 40 can optionally be rendered acidic or basic. For example, the second eluate 40 can be rendered basic by injection of Tris from tank 27. In such embodiments, a second neutralized material 50 is provided.

Optionally, but as shown in FIG. 1, the second neutralized material 50 that includes the antibody of interest, can be transferred to a third column 60 that includes a third adsorbent 62, optionally, without prior filtration of the neutralized material. The third column resin can be optional for either AEX or CHT or CEX, depending upon the specific process results desired. The unfiltered and neutralized material flows through the third column to provide a third eluate 64 that includes the protein of interest. As shown, elution of the third eluate 64 can be performed, optionally, into a holding tank 70. Here, the third eluate can optionally be rendered acidic or basic and/or diluted or concentrated.

The third eluate can be optionally filtered, e.g., using a viral filter 71 and/or a UFDF filtration system 74, and concentrated or diluted to give the final diagnostic or therapeutic antibody product 75 in holding tank 76.

Not filtering (e.g., no UFDF) and/or excluding another complicated manipulation, such as adding salt or a diluting, between column pairs can enable higher throughput and can allow for a continuous process and/or multiple passes through the systems to maximize purity and/or efficiency. Not filtering (e.g., no UFDF) and/or excluding another complicated manipulation can also enable smaller columns, which can lower the usage of expensive adsorbents and can lower the overall cost of the processes. Furthermore, not filtering can eliminate the cost of the filter and hardware associated with the filter. In addition, not filtering and/or otherwise manipulating can reduce holding tank sizes and process time, which can reduce overall cost. In addition, having a continuous process and elimination of UFDF filtering can reduce exposure time of fragile proteins to process conditions. For example, ProA resin costs approximately $9,000 per L, while other resins and ceramics can cost between about $1,000 to about $2,500 per L.

In some embodiments, each column is large enough to provide maximum throughput capacity and economies of scale. For example, each column can define an interior volume of greater than about 200 L, greater than about 500 L, about 1000 L or even greater than about 1500 L.

In embodiments, the systems can process greater than about 200 L of culture per hour, e.g., greater than about 400 L, about 600 L, about 800 L or even greater than about 1500 L per hour.

In some implementations, the culture is provided by cell culture fermentation, e.g., recombinant cell culture fermentation, e.g., CHO fermentation, or is selected and purchased from a supplier.

In some implementations, the systems are capable of handling high titer concentrations, e.g., concentrations of about 5 g/L, greater than about 5 g/L, e.g., greater than about 6, about 7, about 8, about 9, about 10, about 12.5, about 15, about 20 or even greater than about 25 g/L. For example, some of the systems are capable of handling high antibody concentrations and, at the same time, can process greater than about 200 L culture per hour, e.g., greater than about 400 L, about 600 L, about 800 L or even greater than about 1500 L per hour.

In some instances, the first and second adsorbents are different. In instances in which a third column is present, the first adsorbent, second adsorbent and third adsorbents can each be different.

For example, each adsorbent can be or can include a polymeric resin or an inorganic material, such as a ceramic. When a ceramic is utilized, it can be functionalized with, e.g., a hydrophobic and/or hydrophilic group. Mixtures of polymeric resins and inorganic materials can be utilized.

For example, the polymeric resin can be or can include an ion exchange resin, e.g., a cationic, an anionic, or mixed bed ion exchange resin, or the resin can be or can include a hydrophobic charge induction resin. Mixtures of polymeric resins can be utilized.

A specific example of a polymeric resin is MABSELECT™ Protein A resin (ProA), which is available from GE Healthcare. An example of a hydrophobic charge induction resin is 4-mercapto-ethyl-pyridine resin-based MEP HYPERCEL®, which is available from Pall Corporation. A specific example of an anion exchange resin (AEX) is CAPTO™ Adhere, which is available from GE Healthcare. A specific ceramic adsorbent is CHT ceramic hydroxyapatite, which is available from BIO-RAD.

Other polymeric resins and ceramic resins that can be utilized in any column described herein are described in J. Chen et al., J. Chromatogr. A 1177:272-281 (2008), doi: 10.1016/j.chroma.2007.07.083.

In some embodiments, combinations of one or more ProA columns, ion exchange columns, e.g., anionic, cationic or mixed bed columns, and CHT columns are utilized. In other embodiments, combinations of one or more MEP, AEX and CHT columns are utilized.

In some embodiments, a combination of one or more ProA columns, MEP columns and AEX columns, e.g., CAPTO™ Adhere are utilized. For example, the first column can be a ProA column, the second column can be an MEP column and the third column can be an AEX column.

In specific implementations, the system includes three different columns including three different adsorbents. For example, in one implementation, the three columns are ProA (first), MEP (second) and CHT (third). In other implementations, the three columns are ProA (first), MEP (second) and CAPTO™ Adhere (third). In still other implementations, the three columns are MEP (first), CAPTO™ Adhere (second) and CHT (third).

For example, and by reference again to FIG. 1, in a specific implementations, column 1 is ProA, column 2 is MEP and column 3 is CHT; or column 1 is ProA, column 2 is MEP and column 3 is CAPTO™ Adhere; or column 1 is MEP, column 2 is CAPTO™ Adhere and column 3 is CHT.

### EXAMPLES

### Example 1

### ProA-MEP-CHT Production Process for DYAX mAb DX2300

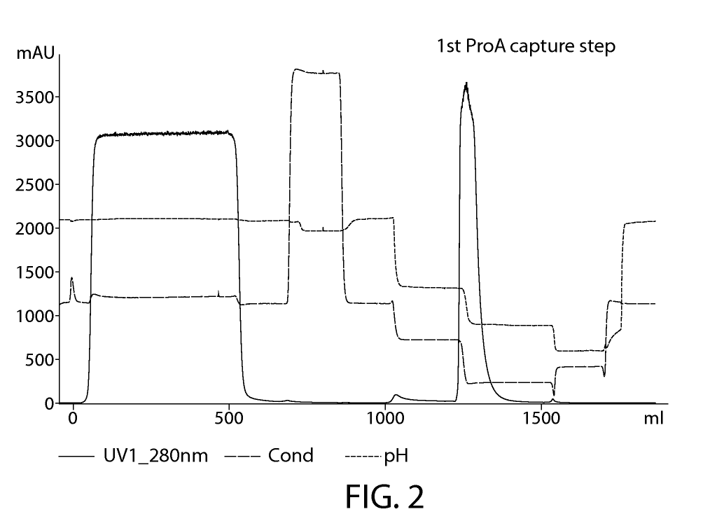

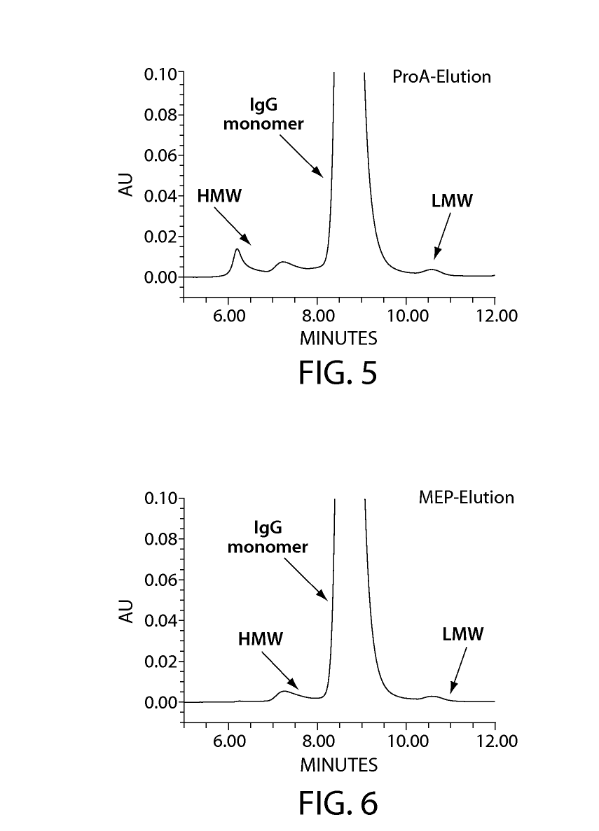

DX2300 mAb was produced from CHO fermentation in a bioreactor. The culture was harvested through a depth filtration process using Millipore D1HC and B1HC depth filters, followed by 0.2 micron filtration. Clarified CHO culture supernatant was then loaded onto a pre-packed ProA affinity column with MABSELECT™ ProA resin from GE Healthcare. DX2300 product captured by the ProA step purification process was eluted under low pH conditions (pH 3.2+/−0.1), and held a low pH conditions for more than 1 hour for viral inactivation. The virus-inactivated material was then neutralized to pH 7.5 using 1M Tris buffer. FIG. 2 shows an LC chromatogram of the eluate, while FIG. 5 shows a SEC-HPLC chromatogram of the eluate.

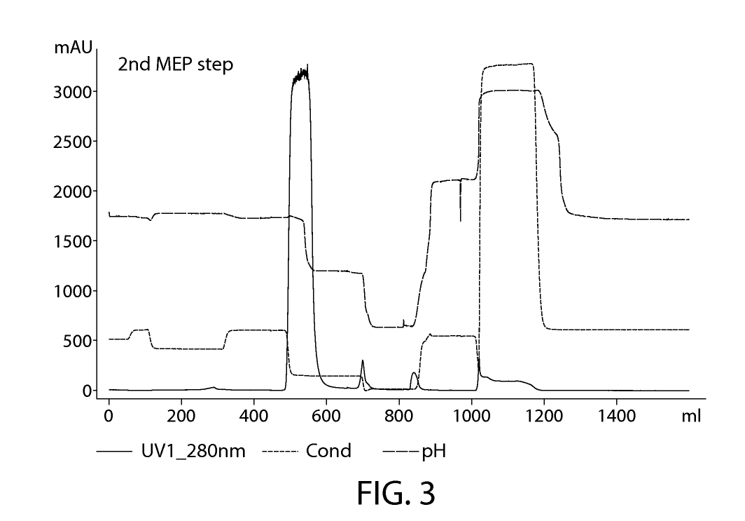

Neutralized ProA elution product was then loaded onto a pre-packed MEP column (without prior filtration) and subjected to a second purification. Post MEP elution material had a pH of about 5.5 and conductivity <4 mS/cm. FIG. 3 shows an LC chromatogram of the eluate, while FIG. 6 shows a SEC-HPLC chromatogram of the eluate.

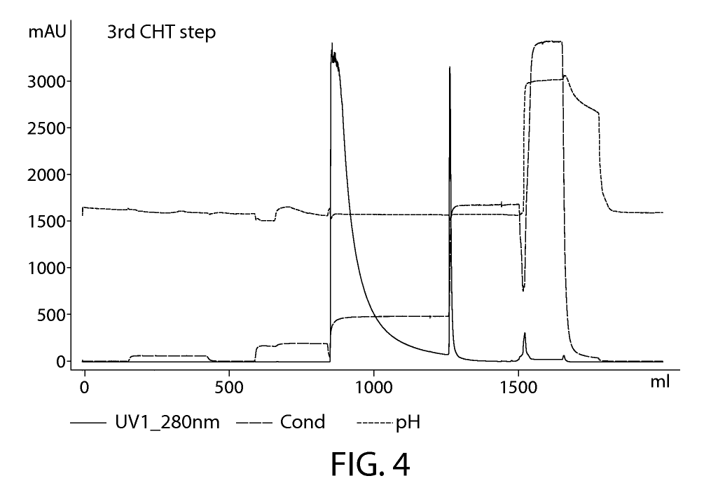

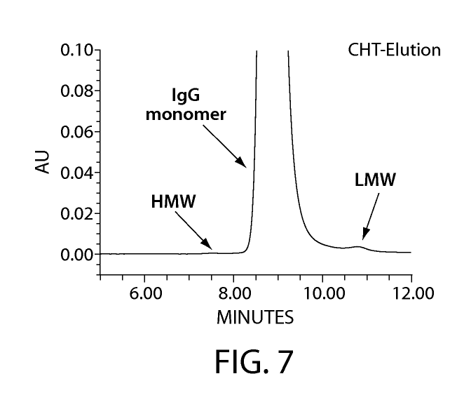

The post MEP product was then loaded onto a pre-packed CHT column (without prior filtration). Only pH adjustment using 1M Tris buffer to pH 6.8 was utilized. Little or no dilution with water was necessary to maintain the conductivity below 4 mS/cm. FIG. 4 shows an LC chromatogram of the eluate, while FIG. 7 shows a SEC-HPLC chromatogram of the eluate.

A comparison of FIGS. 5 to 6 to 7 show the enrichment of IgG monomer and a decrease in HMW and LMW contaminants with each step of the purification process.

After CHT purification, the DX2300 product was filtered using a 20N viral filter and then ultrafiltration/diafiltration to buffer exchange into final formulation buffer with desired product concentration.

Yields for each process step in the ProA-MEP-CHT system of the Example are summarized in TABLE 1. Yields obtained by a conventional process (ProA-UFDF1-AEX-CEX Platform) are also provided for comparison.

TABLE 1

| | |
|---|---|
| ProA-MEP-CHT | ProA-UFDF1-AEX-CEX |
| Platform | Platform |

| Purification Step | Yield | Purification Step | Yield |
|---|---|---|---|
| Harvest | 90% | Harvest | 90% |
| ProA step | 90% | ProA step | 90% |
| MEP step | 85% | UFDF1 step | 90% |
| CHT step | 85% | AEX step | 90% |
| | | CEX step | 90% |
| Viral filtration | 90% | Viral filtration | 90% |
| Final UFDF step | 90% | Final UFDF step | 90% |
| Total DSP | ~50% | Total DSP | ~50% |

Product purity parameters from the ProA-MEP-CHT system in comparison to a conventional ProA-UFDF1-AEX-CEX system are summarized in TABLE 2.

TABLE 2

| | | Conventional |
|---|---|---|
| | ProA-MEP-CHT | ProA-UFDF1-AEX-CEX |
| | System | System |
| Purity % by SEC-HPLC | 99% | 98% |
| HMW % by SEC-HPLC | 0.03% | 1.5% |
| LMW % by SEC-HPLC | 0.48% | 0.29% |
| CHO HCP Level (ppm) | 0.48 ppm | 0.12 ppm |
| Leached ProA level (ppm) | 1.31 ppm | 3 ppm |

This example shows that the purity parameters of the product obtained from the new systems described herein are equivalent to or even better than those obtained for the same product using the conventional ProA-UFDF1-AEX-CEX system. Using the ProA-MEP-CHT platform design, sample manipulation between chromatography processes and decrease production steps was simplified. Impurity deduction (such as HMW % deduction) was also increased to about 90% as compared to about 65-70% with conventional ProA-UFDF1-AEX-CEX platform.

### Example 2

### Mix Mode MEP Unit Operation Elution for mAb DX2400

Based on MEP resin design, the ligand, Mercapto-Ethyl-Pyridine, consists of a hydrophobic tail and an ionizable headgroup with pKa at 4.8. Without being bound by theory, the mechanism of binding antibody molecules is typically such that under conditions where the aromatic pyridine ring is uncharged, IgG binds to the resin through mainly hydrophobic interactions. When buffer pH decreases to below 4.8, the ligand takes on a distinct positive charge. Meanwhile, most of the IgG molecules with relative higher pI would also carry positive charges. As a result, the electrostatic repulsion is induced and antibody is desorbed from the column.

For DX2400, different approaches were designed to elute the product based on dual-mode ligand design. It was discovered that if IgG was eluted mainly through decreasing hydrophobic interaction while the aromatic pyridine ring of the resin's ligand is uncharged, more impurities, in particular for aggregates, were removed.

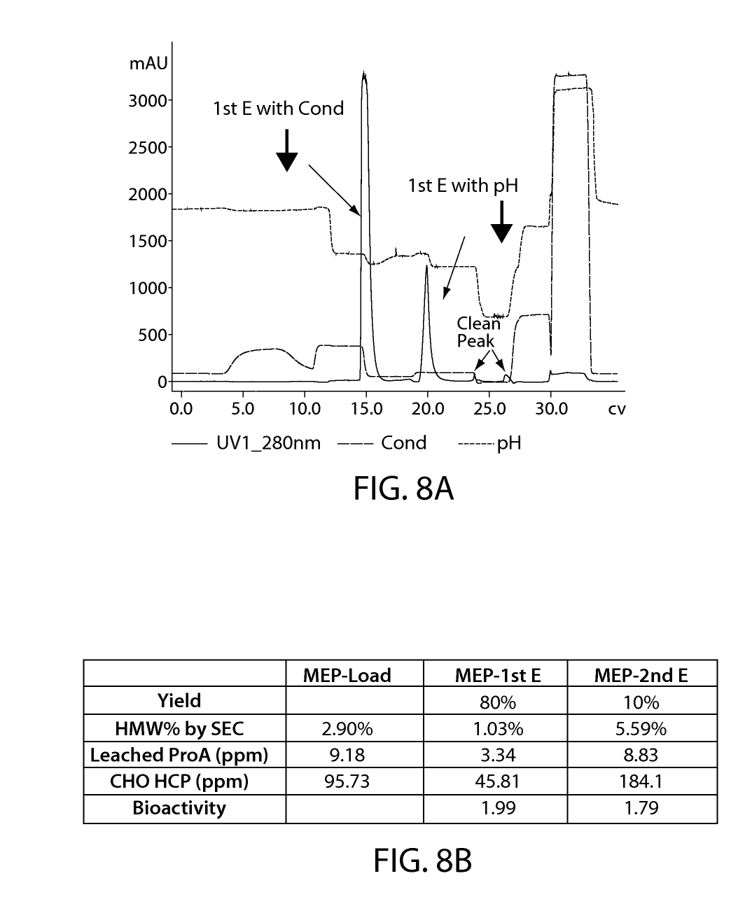

The results are shown in FIGS. 8A and 8B.

FIG. 8A is a LC chromatogram of a DYAX mAb DX2400-rich eluate obtained by passing the ProA eluate through MEP column using dual separation strategies. 1st E with Cond refers to the first elution with conductivity (the conductivity to decreased to less than 4 mS/cm); 2nd E with pH refers to the second elution with a change (lowering) in pH. FIG. 8B is a table of product purity analysis that shows that the first eluate peak derived from dominant HIC strategy separation provides purer antibody product, in terms of aggregates (HMW) level, leached ProA level (ProA ppm) and host CHO contaminated protein level (CHO HCP ppm) as compared to DX2400 purified by a conventional electrostatic repulsive elution approach.

### Other Embodiments

A number of embodiments of the invention have been described. Nevertheless, it will be understood that various modifications may be made without departing from the spirit and scope of the invention.

For example, an eluate can make multiple passes through any one filter.

Each column system can include more than three columns, e.g., 4, 5, 6, 7, 8, 9, 10, 11, 15, or even more than 20 columns.

The columns may be stacked vertically so that each column forms a portion of a large column.

Accordingly, other embodiments are within the scope of the following claims.

### REFERENCES

[1] A. S. Lubiniecki, J. Bioprocess. (2003) 21.
[2] U. Gottschalk, Biopharm. Int. (2006) 8.
[3] G. Jagschies, A. Gronberg, T. Bjorkman, K. Lacki, H. J. Johansson, Biopharm. Int. (2006) 10.
[4] E Li, J. X. Zhou, X. Yang, T. Tressel, B. Lee, J. Bioprocess. (2006) 16.
[5] M. A. Schenerman, B. R. Sunday, S. Kozlowski, K. Webber, H. Gazzano-Santoro, A. Mire-Sluis, Bioprocess Int. (2004) 42.
[6] C. Scott, Bioprocess Int. (2006) 24.
[7] G. Sofer, L. C. Chirica, Biopharm. Int. (2006) 48.
[8] A. Gronberg, E. Monie, M. Murby, G. Rodrigo, E. Wallby, H. Johansson, BioProcess. Int. (2007) 48.
[9] R. Godavarti, Presented at IBC's 16th International Antibody Development & Production Conference, San Diego, Calif., USA, February 2006.
[10] A. H. Guse, A. D. Milton, H. Schulze-Koops, B. Muller, E. Roth, B. Simmer, J. Chromatogr. A 661 (1994) 13.
[11] I. Tornoe, I. L. Titlestad, K. Kejling, K. Erb, H. J. Ditzel, J. C. Jensenius, J. Immunol. Methods 205 (1997) 11.
[12] P. Gagnon, Purification Tools for Monoclonal Antibodies, Validated Biosystems Publish, USA, 1996.
[13] P. Shadle, G. Mills, J. Erickson, R. Scott, T. Smith, U.S. Pat. No. 5,429,746 (1995).
[14] E. H. Rinderknecht, G. A. Zapata, U.S. Pat. No. 7,038,017 (2006).
[15] S. C. Burton, D. R. K. Harding, “Hydrophobic charge induction chromatography: salt independent protein adsorption and facile elution with aqueous buffer”, J. Chromatogr. A, 814 (1998) 71-81.
[16] E. Boschetti, “Antibody separation by hydrophobic charge induction chromatography”, Trends in Biotechnology, 20 (2002), 333-337.
[17] S. C. Burton, D. R. K. Harding, “Salt-independent adsorption chromatography: new broad-spectrum affinity methods for protein capture”, J. Biochem. Biophys. Methods 49 (2001), 257-287.
[18] W. Schwartz, D. Judd, M. Wysocki, L. Guerrier, E. Birck-Wilson, E. Boschetti, “Comparison of hydrophobic charge induction chromatography with affinity chromatography on protein A for harvest and purification of antibodies”, J. Chromatogr. A 908 (2001) 251-263.
[19] S. Ghose, B. Hubbard, S. M. Cramer, “Evaluation and comparison of alternatives to Protein A chromatography mimetic and hydrophobic charge induction chromatographic stationary phases”, J. Chromatogr. A 1122 (2006) 144-152.
[20] G. M. Ferreira, J. Dembecki, K. Patel, A. Arunakumari, “A two-column process to purify antibodies without Protein A”, BioPharm International, May 2007 page 32-44.
[21] M. Rios, “Eluting possibilities with mixed-mode chromatography”, Pharmaceutical Technology, May 2007.

What is claimed is:

1. A method of purifying a protein, the method comprising:
providing a culture comprising a protein;
flowing the culture through a first column comprising a first adsorbent to provide a first eluate comprising the protein; and
flowing the first eluate, or a concentrated or a diluted form thereof, through a second column comprising a second adsorbent without prior ultrafiltration/diafiltration (UFDF) of the first eluate, or the concentrated or the diluted form thereof, to provide a second eluate comprising the protein.

2. The method of claim 1, wherein the method further comprises flowing the second eluate, or a concentrated or a diluted form thereof, through a third column comprising a third adsorbent without prior filtration of the second eluate, or the concentrated or the diluted form thereof, to provide a third eluate comprising the protein.

3. The method of claim 1, wherein the culture is provided by recombinant cell culture fermentation.

4. The method of claim 1, wherein the protein comprises an antibody.

5. The method of claim 1, wherein the culture provide is clarified prior to flowing the culture through the first column, such as by flowing a raw culture through one or more membranes each having pores less than about 1 micron.

6. The method of claim 1, wherein the first and second adsorbents are different.

7. The method of claim 2, wherein the first, second and third adsorbents are different.

8. The method of claim 2, wherein the first adsorbent, second adsorbent and third adsorbents are ProA, MEP and CHT, respectively.

9. The method claim 1, wherein prior to flowing the first eluate, or the concentrated or the diluted form thereof, through the second column, a pH of the first eluate, or the concentrated or diluted form thereof, is changed by adding an acid, a base or a buffer, to the first eluate, or the concentrated or the diluted form thereof.

10. The method of claim 2, wherein prior to flowing the second eluate, or the concentrated or the diluted form thereof, through the third column, a pH of the second eluate, or the concentrated or diluted form thereof, is changed by adding an acid, a base or a buffer to the second eluate, or the concentrated or the diluted form thereof.

11. The method of claim 1, wherein the first or second column has a volume of about 200 L or more.

12. A protein purification system comprising one or more columns, each comprising an adsorbent therein, wherein the protein purification system is capable of accepting a culture having a protein concentration of greater than about 5 g/L, and with an overall yield of greater than about forty percent.

13. The protein purification system of claim 12, wherein the protein purification system is capable of purifying the protein to an extent of greater than about ninety-five percent, as measured using SEC-HPLC.

14. The protein purification system of claim 12, wherein the protein purification system is capable purifying the protein to an extent of greater than about ninety-nine percent with an overall yield of greater than about fifty percent.

15. The protein purification system of claim 12, wherein the protein purification system is capable of processing greater than about 200 L per hour of the culture.

16. A protein purification system comprising one or more columns, each comprising an adsorbent therein, wherein the protein purification system is capable of processing greater than about 200 L per hour of a culture having a protein concentration of greater than about 5 g/L.

17. The protein purification system of claim 16, wherein the protein purification system is capable of processing greater than about 500 L of culture per hour.

18. The protein purification system of claim 16, wherein the protein purification system is capable purifying the protein to an extent of greater than about ninety-nine percent with an overall yield of greater than about fifty percent.

19. A protein purification system comprising one or more columns, each comprising an adsorbent therein, wherein each column comprises less than about 250 L of adsorbent, and wherein the protein purification system is capable of accepting a culture having a protein concentration of greater than about 5 g/L.

20. The protein purification system of claim 19, wherein the system is capable of purifying the protein to an extent of greater than about ninety-five percent with an overall yield of greater than about forty percent.
